(19) United States
(12) Patent Application Publication
(10) Pub. No.: US 2010/0159488 A1
(43) Pub. Date: Jun. 24, 2010

(54) METHOD FOR THE MULTIPLEX SEROLOGICAL DIAGNOSIS IN VITRO OF SPIROCHETE INFECTIONS

(75) Inventors: Michel Drancourt, Marseilles (FR); Didier Raoult, Marseille (FR)

Correspondence Address: DENNISON, SCHULTZ & MACDONALD, 1727 KING STREET, SUITE 105, ALEXANDRIA, VA, 22314, US

(73) Assignee: INODIAG, Signes (FR)

(21) Appl. No.: 12/664,158

(22) PCT Filed: Jul. 3, 2008
(86) PCT No.: PCT/EP08/58583

§ 371 Date: Dec. 11, 2009

(30) Foreign Application Priority Data
Jul. 4, 2007 (FR) 0756269

(51) Int. Cl. G01N 33/53

(52) U.S. Cl. 435/7.92

(57) ABSTRACT

A method for the serological diagnosis, in vitro, of an infection with a spirochete bacterium which is pathogenic in humans, chosen from bacteria of the Borrelia, Leptospira and Treponema genera, by carrying out at least one of 1) an assay of IgG-type and/or IgM-type antibodies against at least one tick-borne Borrelia bacterium, chosen from Borrelia duttonii and Borrelia crocidurae, and 2) an assay of IgM-type antibodies against the louse-borne Borrelia bacterium Borrelia recurrentis.

The present invention relates to a method for the serological diagnosis in vitro, of an infection with a spirochete bacterium which is pathogenic in humans, chosen from the bacteria of the Borrelia, Leptospira and Treponema genera, by indirect immunodetection.

More particularly, the present invention relates to a method for the serological diagnosis, in vitro, of infections with the following bacteria: Treponema pallidum, agent which causes syphilis; Leptospira interrogans, agent which causes leptospirosis; Borrelia burgdorferi, agent which causes Lyme disease; Borrelia recurrentis, agent which causes louse-borne relapsing fever; Borrelia spp., agents which cause tick-borne relapsing fever, other than Borrelia burgdorferi, such as Borrelia duttonii or Borrelia crocidurae.

The abovementioned bacteria are responsible for many diseases which can present in the form of an isolated fever or in the form of febrile relapses or chronic manifestations. These diseases can be contracted by contact with soiled water (Leptospira), by sexual contact (Treponema), or through tick or louse bites (Borrelia). Each spirochete infection can present specific clinical signs, but spirochete infections are essentially infections responsible for nonspecific signs, in the form of fever and a worsening of the general condition over the course of relapsing fevers and in the form of neurological manifestations (neuritis, meningitis, encephalitis) or opthalmological manifestations (uveitis) during which obtaining the patient's history and clinical examination of the patient are not sufficient to establish the etiology of the infection. Recourse to laboratory tests is then essential [Raoult D, Hechemy K E, Lecam C, Enea M, Tamalet J. Crossed reactions in Lyme disease. Value of the Western blot. Press. Med. 1988; 17:485].

Among the forms of borreliosis, 5 bacteria can be distinguished which are responsible for diseases widespread in humans, namely 3 types of bacteria and diseases:
Borrelia burgdorferi which is prevalent in the USA and in Europe, responsible for Lyme disease,
the other tick-borne Borrelia, namely Borrelia hermsii, Borrelia duttonii, Borrelia crocidurae, and Borrelia valaisiana, responsible for forms of borreliosis other than Lyme disease, prevalent in tropical countries and particularly in Africa, and
a louse-borne Borrelia, Borrelia recurrentis, which is prevalent in populations at risk in poor and cold countries, and in particular for individuals living under insufficient sanitary conditions.

Leptospirosis is a disease associated with Leptospira interrogans bacteria. Leptospira biflexa bacteria are not pathogenic in humans, but are nevertheless used as a reference antigen for detecting the disease since Leptospira biflexa induces antibodies in humans with cross-reactions in sera from patients infected with L. interrogans [Levett, P. N., Leptospira and Leptonema. In: Manual of Clinical Microbiology 8th Edition. Murray P R, Baron E J, Jorgensen J H, Pfaller M A, Yolken R H (Eds). ASM Press, Washington D.C., 2003, pp. 929-936].

These diseases associated with spirochete bacteria present, as mentioned above, certain common clinical signs such as meningitis, encephalitis, polyneuritis or polyarthritis. In the case of borreliosis, recurring fevers also commonly occur. However, these clinical signs are not specific for diseases associated with spirochete bacteria. Furthermore, the treatments are different depending on the type of spirochete bacteria, whether between the various bacterial genera Borrelia, Leptospira and Treponema, for the three categories of borreliosis mentioned above, or even for the various species of Borrelia and Leptospira bacteria. Finally, these pathogenic spirochete bacteria give rise to infections inside the eye, called uveitis, but which may be due to other types of bacteria (nonspirochete bacteria). In the Treponema genus, only Treponema pallidum is pathogenic in humans.

Direct diagnosis of these diseases is not easy. This is because these bacteria are fastidious bacteria, the isolation and culture of which from clinical samples take a long time (several weeks) and are not very productive [Levett, P. N., Leptospira and Leptonema. In: Manual of Clinical Microbiology 8th Edition. Murray P R, Baron E J, Jorgensen J H, Pfaller M A, Yolken R H (Eds). ASM Press, Washington D.C., 2003, pp. 929-936] [Wilske B. and Schriefer M E. Borrelia. In: Manual of Clinical Microbiology 8th Edition. Murray P R, Baron E J, Jorgensen J H, Pfaller M A, Yolken R H (Eds). ASM Press, Washington D.C., 2003, pp. 937-954], or even impossible in the case of T. pallidum, which can only be isolated on animals (testes from rabbit and nine-banded armadillo) [Norris S J. et al. Treponema and other human host-associated spirochetes, In: Manual of Clinical Microbiology 8th Edition. Murray P R, Baron E J, Jorgensen J H, Pfaller M A, Yolken R H (Eds). ASM Press, Washington D.C., 2003 pp. 955-971]. Detection of the DNA by molecular biology techniques also has a low sensitivity, such that this is not a question of diagnostic techniques for leptospirosis [Levett P. N. In: Manual of Clinical Microbiology 8th Edition. Murray P R, Baron E J, Jorgensen J H, Pfaller M A, Yolken R H (Eds). ASM Press, Washington D.C., 2003], or experimental techniques for detecting T. pallidum [Norris S J. et al. Treponema and other human host-associated spirochetes, In: Manual of Clinical Microbiology 8th Edition. Murray P R, Baron E J, Jorgensen J H, Pfaller M A, Yolken R H (Eds). ASM Press, Washington D.C., 2003 pp. 955-971]. Molecular detection is used in some specialized laboratories for the diagnosis of Lyme disease (Borrelia burgdorferi) in the case of negative isolation and serology [Wilske B. and Schriefer M E. Borrelia. In: Manual of Clinical Microbiology 8th Edition. Murray P R, Baron E J, Jorgensen J H, Pfaller M A, Yolken R H (Eds). ASM Press, Washington D.C., 2003, pp. 937-954]. Blood smears are used for tick-borne forms of borreliosis, but are not very sensitive [Vial L, Diatta G, Tall A, Ba el H, Bouganali H, Durand P, Sokhna C, Rogier C, Renaud F, Trape J F. Incidence of tick-borne relapsing fever in west Africa: longitudinal study. Lancet. 2006 Jul. 1; 368 (9529):37-43]. Overall, it is therefore the serology which constitutes the usual mode of diagnosis for human spirochete infections, that is widespread in medical biology laboratories [Levett P. N. In: Manual of Clinical Microbiology 8th Edition. Murray P R, Baron E J, Jorgensen J H, Pfaller M A, Yolken R H (Eds). ASM Press, Washington D.C., 2003] [Wilske B. and Schriefer M E Borrelia. In: Manual of Clinical Microbiology 8th Edition. Murray P R, Baron E J, Jorgensen J H, Pfaller M A, Yolken R H (Eds). ASM Press, Washington D.C., 2003, pp. 937-954] [Norris S J. et al. Treponema and other human host-associated spirochetes, In: Manual of Clinical Microbiology 8th Edition. Murray P R, Baron E J, Jorgensen J H, Pfaller M A, Yolken R H (Eds). ASM Press, Washington D.C., 2003 pp. 955-971].

The serology of spirochete infections consists in searching, in a blood sample from the patient, for the presence of antibodies directed against one of the spirochete species. However, these bacteria comprise cross-reactions with Borrelia burgdorferi, which means that, when a patient is infected with one of these bacteria, said patient may have nonspecific antibodies that react against the Borrelia burgdorferi bacterium (see table 1 at the end of the description). Currently, when it is desired to diagnose Lyme disease, a serological reaction precisely against the Borrelia burgdorferi bacterium, responsible for this disease, is demanded without testing all the bacteria capable of giving comparable manifestations and having common antigens.

The presence of serological reactions that are falsely positive for Lyme disease (Borrelia burgdorferi) has been shown by microimmuno-fluorescence and ELISA in the course of syphilis (Treponema pallidum) [Raoult D, Hechemy K E, Baranton G. Cross-reaction with Borrelia burgdorferi antigen of sera from patients with human immunodeficiency virus infection, syphilis, and leptospirosis. J. Clin. Microbiol. 1989; 27:2152-5] [Raoult D, Hechemy K E, Lecam C, Enea M, Tamalet J. Crossed reactions in Lyme disease. Value of the Western blot. Press. Med. 1988; 17:485] [Magnarelli L A, Anderson J F, Johson R C. Cross-reactivity in serological tests for Lyme disease and other spirochetal infections. J. Infect. Dis. 1987; 156:183-188], in the course of leptospirosis (Leptospira spp.) [Raoult D, Hechemy K E, Lecam C, Enea M, Tamalet J. Crossed reactions in Lyme disease. Value of the Western blot. Press. Med. 1988; 17:485] [Magnarelli L A, Anderson J F, Johson R C. Cross-reactivity in serological tests for Lyme disease and other spirochetal infections. J. Infect. Dis. 1987; 156:183-188], and in patients presenting a clinical picture of another tick-borne borreliosis in the USA (Borrelia hermsii) and louse-borne borreliosis in Ethiopia, Borrelia recurrentis, but only in the case of the assaying of IgM [Magnarelli L A, Anderson J F, Johson R C. Cross-reactivity in serological tests for Lyme disease and other spirochetal infections. J. Infect. Dis. 1987; 156:183-188].

The only methods currently available for overcoming the serological cross-reactions between spirochetes are cross absorptions [Magnarelli L A, Anderson J F, Johson R C. Cross-reactivity in serological tests for Lyme disease and other spirochetal infections. J. Infect. Dis. 1987; 156:183-188] and the Western blotting technique, which consists in reacting the patient's serum against the antigenic proteins of the spirochete, separated from one another by electrophoresis. However this is a long technique which uses 200 μL of serum, and which is available only for the serological diagnosis of Lyme disease (Borrelia burgdorferi).

There is therefore currently no technique for demonstrating serological cross-reactions between all the spirochetes, and therefore for reasoned interpretation of the serology of spirochete infections.

The aim of the present invention is to provide a method for serological diagnosis, in vitro, by indirect immunodetection which is of use in the case of patients suspected of being infected with any pathogenic spirochete bacterium of the Borrelia spp., Leptospira spp. and Treponema pallidum genera, which makes it possible to provide the maximum amount of information in relation to the presence of an infection with a spirochete bacterium in general and more specifically in relation to the main spirochete bacteria responsible for diseases most widespread in humans, mentioned above.

Another aim of the present invention is to provide a method for diagnosis which makes it possible to determine whether the antibody of the serum having reacted with the bacterial antigen of the bacterium tested is actually an antibody specific for said bacterium or an antibody for another spirochete bacterium, or the like.

Another aim of the present invention is to provide a method for diagnosis which is reliable, simple and rapid to use.

To do this, the inventors have developed, for the first time, indirect-immunodetection assays for serological reaction between
a tick-borne Borrelia bacterium, other than Borrelia burgdorferi, in particular Borrelia duttonii or Borrelia crocidurae, deposited on a solid support and patient immunoglobulins of IgG and IgM type against said tick-borne Borrelia species, in particular Borrelia duttonii or Borrelia crocidurae, and
a louse-borne Borrelia bacterium, namely Borrelia recurrentis, deposited on a solid support and patient immunoglobulins of IgM type against Borrelia recurrentis.

The inventors have also demonstrated the existence of cross-reactions in pairs between the bacteria Borrelia burgdorferi, Borrelia recurrentis, and tick-borne Borrelia other than Borrelia burgdorferi, such as Borrelia duttonii or Borrelia crocidurae, and also the bacteria Leptospira biflexa serovar Patoc (nonpathogenic leptospira) and Leptospira interrogans serovar Icterohaemorrhagiae (pathogenic leptospira) and the bacterium Treponema pallidum (see table 2 at the end of the description).

The present invention provides a method consisting in testing, in a single series of multiplexed serological reactions, according to the method described in WO 2005/064340 in the name of the applicant, namely on a single solid support, on the same serum sample, a plurality of spirochete bacteria representative of all the spirochete bacteria in parallel, which makes it possible to interpret the results in terms of specificity of serological reaction by comparison of the level of the signal obtained for each of the spirochetes, determined over the course of this multiplexed serology.

More specifically, the present invention provides a method for the serological diagnosis, in vitro, of an infection with a spirochete bacterium which is pathogenic in humans, chosen from bacteria of the Borrelia, Leptospira and Treponema genera, by indirect immunodetection, characterized in that the following steps are carried out, comprising:

1/bringing a same patient serum sample into contact with

a/ —a first detection substance and/or respectively second detection substance constituted of an antibody which reacts only with an immunoglobulin of the species of the patient of IgG type and/or respectively IgM type and

b/ —a plurality of bacterial antigens deposited on several zones of a same solid support, comprising:
a plurality of bacterial antigens of bacteria, of the Borrelia genus, of different species comprising at least the bacteria Borrelia burgdorferi, Borrelia recurrentis and a tick-borne Borrelia species other than Borrelia burgdorferi, chosen from Borrelia duttonii and Borrelia crocidurae, said bacterial antigens being constituted respectively of at least fractions of bacteria of said species, each said fraction of said bacterium comprising at least one specific antigen and one nonspecific antigen of said species of bacterium, preferably whole bacteria, and
a plurality of bacterial antigens of bacteria, of the Leptospira genus, of different species comprising at least one commensal species which is not pathogenic in humans and one pathogenic bacterium of the Leptospira interrogans species, said bacterial antigens being constituted respectively of at least fractions of bacteria of said species, each said fraction of said bacterium comprising at least one specific antigen and one nonspecific antigen of said bacterium, preferably whole bacteria, and
at least one bacterial antigen of the bacterium Treponema pallidum, preferably a specific bacterial antigen of Treponema pallidum, and

2/detecting and preferably quantifying the serological reactions of IgG-type and/or IgM-type immunoglobulins present, as appropriate, in said patient serum, which react with at least one of said bacterial antigens, by detecting and preferably quantifying a signal emitted by a ternary complex of an immunological reaction between (a) said bacterial antigen(s), (b) a said immunoglobulin which reacts with at least one of said bacterial antigens, said immunoglobulin being an IgG-type immunoglobulin for detecting and assaying IgG, and/or respectively a said IgM-type immunoglobulin for detecting and assaying IgM, and (c) a said first detection substance for detecting and assaying IgG and/or respectively second detection substance for detecting and assaying IgM.

The term “commensal bacterium” is herein intended to mean a bacterium of a spirochete species normally present in contact with the mucosae of humans, comprising cultured spirochete species and species not yet cultured but characterized by molecular analyses such as those targeting the sequence of the universal 16S rRNA gene. Such species have in particular been characterized in the oral cavity of humans and the intestinal flora thereof.

The quantification of the serological reactions is carried out by measuring the signal emitted by the labeling elements incorporated into said detection substances, after the latter have reacted with the solid support, as will be explained hereinafter.

More particularly, the pluralities of said bacteria are constituted:
for bacteria of the Borrelia genus of different species, of the three bacteria Borrelia burgdorferi, Borrelia recurrentis and Borrelia duttonii, and
for bacteria of the Leptospira genus, of the two bacteria Leptospira biflexa Patoc and Leptospira interrogans which are the bacteria most representative of the two pathogenic and nonpathogenic species of Leptospira.

Even more particularly, at least one assay of the IgM type antibodies against the louse-borne Borrelia bacterium Borrelia recurrentis is carried out.

Even more particularly, the bacterial antigen of the bacterium Treponema pallidum is the p17 antigen, and preferably said solid support also comprises an additional zone on which cardiolipid is deposited. The p17 antigen reveals that the patient has actually been in contact with Treponema pallidum, the agent responsible for syphilis, whereas the presence of anti-cardiolipid antibodies is a specific marker for activity of the Treponema pallidum infection, indicating active syphilis.

The present invention therefore provides a method involving multiplexed serological reactions including a plurality of human spirochetes, making it possible to detect the antibodies specific to each of the spirochetes tested, but also the common antibodies without diagnostic value.

The use of antigenic fractions with nonspecific antigens and specific antigens for the antigens of the bacteria Borrelia spp. and Leptospira spp. in question, and also the use of a plurality of species of Borrelia bacteria corresponding to the three main diseases in humans caused by Borrelia, and of a plurality of Leptospira spp. species with a Leptospira species which is pathogenic in humans and a Leptospira species which is nonpathogenic in humans, make it possible to provide a more relevant and more accurate diagnosis than the current reference methods, as will be shown hereinafter, in accordance with the aim of the present invention.

On the basis of numerous tests carried out on more than 200 sera, the inventors have been able to establish that:
firstly, a plurality of at least six said bacterial antigens of spirochete bacteria according to the invention make it possible to validly reflect all the cross-reactions that may occur between the various bacteria of the Borrelia, Leptospira and Treponema genera, and
secondly, the comparison of the levels of the signals of the reactions of the detection substances with the IgGs and/or IgMs of the patients, which react with said particular bacterial antigens deposited on the solid support, illustrating the most widespread diseases, makes it possible to come to a conclusion as to the specificity of these reactions. In the case of reactions with specific antibodies, the signal accumulates with that related to the binding of the nonspecific antibodies, and the signal is stronger than in the case of cross-reactions involving only the nonspecific antigens.

As the tests carried out currently stand, the inventors prefer to interpret the reactions appearing to be specific for Borrelia duttonii or B. crocidurae and Leptospira biflexa as corresponding more generally to a tick-borne Borrelia other than B. burgdorferi such as, in particular, B. hermsii and, respectively, a nonpathogenic commensal Leptospira, which are very widespread in humans.

Thus, more particularly, the method for diagnosis according to the invention makes it possible to determine:
the presence of a particular bacterium of a said species Borrelia burgdorferi or Borrelia recurrentis or tick-borne Borrelia other than Borrelia burgdorferi, such as Borrelia duttonii, or a pathogenic bacterial species Leptospira interrogans or a nonpathogenic leptospiral bacterial species such as Leptospira biflexa, or the presence of Treponema pallidum, if there is observed a plurality of signals from reactions with said bacterial antigens of bacteria of a same species, deposited on the solid support, preferably, as appropriate, of bacteria of the same genus, with a signal that is of significantly greater strength for said particular bacterium than for the others, and
the presence of a spirochete bacterium of the Borrelia genus or of the Leptospira genus, of a species other than those of the bacteria of said corresponding bacterial antigens deposited on the solid support, without determination of the species and, as appropriate, of the serovar, it there is observed a plurality of signals from reactions with said baterial antigens for bacteria of the same genus Borrelia or Leptospira, without a signal that is of significantly greater strength than the others for a said particular bacterium, and without a signal from reaction with a bacterial agent of a bacterium of the other genus Leptospira or, respectively, Borrelia, and
the presence of a spirochete bacterium of the Borrelia genus or of the Leptospira genus, other than those of the bacteria of said corresponding bacterial antigens deposited on the solid support, without determination of the genus Borrelia or Leptospira, if there is observed a plurality of signals from reactions with said bacterial antigens for bacteria of the two genera Borrelia and Leptospira and, optionally, in addition, with Treponema pallidum, without a signal that is of significantly greater strength (greater intensity) than the others for a said particular bacterium, and
the absence of any spirochete bacterium of the Borrelia or Leptospira genus, if no signal is observed with any of said bacterial antigens of bacteria of the Borrelia and Leptospira genera, and
the absence of Treponema pallidum, if no signal from reactions with said corresponding bacterial antigen is observed.

It is understood that it is therefore possible to determine:
the absence of a particular bacterium of a said species Borrelia burgdorferi or Borrelia recurrentis or another tick-borne Borrelia other than Borrelia burgdorferi, such as Borrelia duttonii, or a pathogenic bacterial species Leptospira interrogans or a nonpathogenic Leptospiral bacterial species such as Leptospira biflexa, or the absence of Treponema pallidum, if there is observed a plurality of signals from reactions with said corresponding bacterial antigens deposited on the solid support, without a reaction signal that is of significantly greater strength for said particular bacterium than for the others, and
the absence of any spirochete bacterium of the Borrelia or Leptospira genus, or of any Treponema pallidum, if no signal is observed with any of said bacterial antigens of bacteria of the Borrelia, Leptospira and Treponema genera.

More particularly, the presence of an active Treponema pallidum bacterium is determined if signals from reaction with said bacterial antigen of Treponema and with the cardiolipid are observed, said signals preferably being stronger than signals from cross-reactions with other said bacterial antigens, and the absence of an active Treponema pallidum bacterium is determined if no signal from reaction with the cardiolipid is obtained.

In one preferred embodiment of the method according to the invention:

1—Detection signal strength (intensity) cut-offs are determined for each of said bacterial antigens as deposited on said solid support, said cut-offs comprising:

a) a so-called “specificity” cut-off, starting from which it is considered that the signal with a said bacterial antigen may include the signal from a reaction with a said specific antigen, and

b) a so-called nonspecific “sensitivity” cut-off, such that
if the signal has a strength between those of said specificity cut-off and said sensitivity cut-off, it is considered that it may include a reaction with a said nonspecific antigen, and does not include a reaction with a said specific antigen, and
if the signal has a strength below that of said sensitivity cut-off, it is considered to be not significant, and

2—The following are determined:

a) the presence of a bacterium of the Borrelia genus or of the Leptospira genus, of a species other than those of the bacteria of said bacterial antigens deposited on the solid support, with determination of the genus but without determination of the species and, as appropriate, of the serovar, if there is observed a plurality of signals from reactions with said bacterial antigens of bacteria of the same genus Borrelia or Leptospira, deposited on the solid support, said signals having strengths greater than or equal to that of said sensitivity cut-off and below that of said specificity cut-off, no signal from a reaction with a said bacterial antigen deposited on the solid support having an intensity greater than or equal to that of said specificity cut-off, and no signal from a reaction with a said bacterial antigen of the other genus Leptospira or, respectively, Borrelia, deposited on the solid support, having a strength greater than or equal to that of said sensitivity cut-off, and

b) the presence of a bacterium of the Borrelia genus or of the Leptospira genus, and of a species other than those of the bacteria of said bacterial antigens deposited on the solid support, without determination of the genus or of the species, or, as appropriate, of the serovar, if there is observed a plurality of signals from reactions with said bacterial antigens of bacteria of the two genera Borrelia and Leptospira, deposited on the solid support, said signals having strengths greater than or equal to that of said sensitivity cut-off and below that of said specificity cut-off, and no signal from a reaction with a said bacterial antigen deposited on the solid support having a strength greater than or equal to that of said specificity cut-off, and

c) the presence of a particular bacterium of a said species Borrelia burgdorferi or Borrelia recurrentis or a tick-borne Borrelia other than Borrelia burgdorferi, such as Borrelia duttonii or Borrelia crocidurae, or a pathogenic bacterial species Leptospira interrogans or a nonpathogenic Leptospiral bacterial species such as Leptospira biflexa, or the presence of Treponema pallidum, if there is observed a single signal from a reaction with a said bacterial agent of a particular bacterium of the same species, deposited on the solid support, which has a strength greater than or equal to that of the specificity cut-off, and, optionally, reaction signals having strengths below the specificity cut-offs for the reactions with the other bacterial agents deposited on the solid support, and

d) the presence of a Treponema pallidum bacterium if there is observed a signal from a reaction with the specific antigen of Treponema pallidum, deposited on the solid support, which has a strength greater than or equal to that of said specificity cut-off for reaction with a said bacterial antigen of a said particular bacterium, and

e) the presence of an active Treponema pallidum bacterium if, in addition, there is observed a signal from a reaction with the cardiolipid, which has a strength greater than that of the sensitivity cut-off for said cardiolipid.

It is understood that it is therefore possible to determine:
the absence of any spirochete bacterium of some Borrelia or Leptospira genus if no signal from a reaction with a bacterial antigen corresponding to the Borrelia and Leptospira bacteria, which has a strength greater than or equal to that of said sensitivity cut-off, is observed, and
the absence of any spirochete bacterium of some Borrelia or Leptospira genus or Treponema pallidum, if no signal from a reaction, which has a strength greater than or equal to that of said sensitivity cut-off, is observed.

In certain cases, in step 1—, the two specificity and sensitivity cut-offs may be the same, such that, in step 2—, it is not possible to determine a signal having an intermediate strength between the sensitivity cut-off and the specificity cut-off, and only the presence of a particular bacterium is determined in accordance with the wording of paragraphs c), d) and e) of step 2— above.

The present invention makes it possible, for the first time, to test at the same time:

(1) the agent which causes louse-borne relapsing fever, Borrelia recurrentis, this disease being transmitted by lice, constantly on the increase in terms of both IgG and IgM,

(2) the most common borreliosis in East Africa, due to B. duttonii, transmitted by ticks, or B. crocidurae more common in West Africa [Vial L, Diatta G, Tall A, Ba el H, Bouganali H, Durand P, Sokhna C, Rogier C, Renaud F, Trape J F. Incidence of tick-borne relapsing fever in west Africa: longitudinal study. Lancet. 2006; 368:37-43], the inventors having demonstrated cross-reactions between B. duttonii and B. crocidurae,

(3) Treponema pallidum, the agent which causes syphilis,

(4) Borrelia burgdorferi, the agent which causes Lyme disease,

(5) Leptospira interrogans serovar Icterohaemorrhagiae and Leptospira biflexa serovar Patoc, which are the species representative of the two major groups of pathogenic and nonpathogenic Leptospira.

The present invention allows a rapid test with quantification for a single dilution, more particularly for a serum dilution to 1/16th.

According to the present invention, a novel approach for diagnosis is therefore carried out by serological group and not by limiting the question serologically posed to the problem of the diagnosis of all particular forms of spirochetosis, and by replacing all the prior serological techniques used with a single technique which performs both the screening and the diagnosis on a single titer.

Advantageously, said first and/or second detection substances comprise said first and/or respectively second labeling elements emitting signals that can be distinguished.

More particularly, said first and/or second detection substances are goat or chicken immunoglobulins, which are respectively anti-IgG and/or anti-IgM.

In one preferred embodiment, said detection substances comprise a fluorescent label.

The expression “fluorescent labeling” means that the detection substance, in particular the secondary detection antibody, has been rendered fluorescent by coupling or complexation with a suitable fluorescent substance such as fluorescein iso(thio)cyanate, i.e. a substance which emits a detectable radiation after illumination thereof, each said fluorescent substance being characterized by the wavelength at which it should be illuminated (excitation wavelength) and the wavelength of the radiation that it emits (emission wavelength).

As fluorescent labeling substance, mention may in particular be made of fluorescein, coumarin, cyanin and analogs and derivatives of these substances known to those skilled in the art.

When a fluorescent substance is used, the fluorescence associated with the sample tested is read directly on a suitable instrument capable of detecting the radiation at the emission wavelength and of quantifying it.

Such instruments are known to those skilled in the art. The quantification is carried out by comparison of the fluorescent signal emitted by the reaction complex [antigen-specific antibody-detection substance] of the serum tested, with a reference curve obtained by calibration using control sera containing a known concentration of antibody to be detected.

Fluorescent labeling is particularly advantageous in the case of determining the specificity of a reaction insofar as it is essential to obtain an accurate and reliable quantification of the concentration of said specific antibodies, which can be obtained by virtue of the fluorescent signals, the strength of which is directly proportional to the amount of fluorescent molecules emitting the signal.

Preferably therefore, said first and/or second detection substances comprise said first and/or respectively second labeling elements emitting fluorescent signals at different excitation wavelengths that can be quantified by automated reading of the strength of the fluorescent signal emitted by said fluorescent labeling element, by means of a suitable reading instrument capable of quantifying it.

As bacterial antigens, antigens constituted of whole bacteria or of bacterial fractions or fragments comprising one or more antigens are used. It is in fact possible to mechanically fragment the bacterium by mechanical agitation or by sonication, for example, or else to fragment the bacterium by means of an enzymatic method in order to obtain a fraction thereof which conserves the antigens that form the basis of the serological reaction which is the subject of the present invention. The fractions thus obtained are separated or else purified from the other constituents of the microorganism and from its culture medium by means of a suitable method, for example by centrifugation or by filtration. The term “antigenic fraction” or “purified antigen” is then used. The whole bacterium or any bacterial fraction is hereinafter referred to as “particulate or corpuscular antigen” in that the bacterium, or a fraction thereof, cannot be solubilized by dissolution, but only suspended in a suitable fluid. The bacteria or fraction thereof remain visible as individualizable particles by microscopic observation using an optical microscope or an electron microscope, for example.

The present invention makes it possible to directly measure the concentration of specific antibodies which, in the reference serology method by immunofluorescence, is expressed as the inverse of the highest dilution giving a positive signal. There is in fact, in the concentration range of the specific antibodies measured during spirochete infections, a linearity between the amount of fluorescence and the antibody concentration. It is therefore possible to calibrate, on the basis of sera having a titer that is known through the reference method, the instrument in order to detect positive sera with respect to negative sera. Since the positivity cut-off with the reference method is 1/128, a serum dilution to 1/16 can be used. This dilution is advantageous since it corresponds to the dilution used in general on this type of fluorescence-reading instrument.

According to the method of the invention, the antigen is thus deposited, as appropriate, on a solid support of the glass slide or microtitration plate type for carrying out detection techniques by immunodetection, in particular immunofluorescence, or by an enzymatic technique, in particular of the ELISA type. These detection techniques are well known to those skilled in the art, and comprise the successive steps of:

1. decomplementing the serum by heating at 56° C. for 30 minutes,

2. bringing the antigen corresponding to the infectious bacterial agent, attached to a solid support, into contact with the patient serum, then incubating under conditions of time, temperature, hygrometry, mechanical agitation and ionic strength of the medium which allow the antigen-antibody reaction,

3. carefully and thoroughly washing in order to remove the excess patient serum not bound to the solid support,

4. applying a secondary detection antibody which is an animal immunoglobulin directed against the immunoglobulins of the species of the patient under consideration, for example in the case of a human patient, an anti-human immunoglobulin goat immunoglobulin conjugated to a fluorochrome substance, generally fluorescein iso(thio)cyanate, or an enzyme, generally a peroxidase, and incubating under conditions of time, temperature, hygrometry, mechanical agitation and ionic strength of the medium which allow the antigen-antibody reaction,

5. carefully and thoroughly washing in order to remove the excess, unbound, labeled immunoglobulin,

6. detecting a reaction by reading, using a suitable instrument according to the label, such as a fluorescence microscope or a microarray reader for the indirect immunofluorescence technique, or an optical density reader for the enzymatic detection technique of the ELISA type. The reaction is read with the naked eye or after digital acquisition of the gel and analysis using densitometry software or any software suitable for image processing.

The present invention relates more particularly to the detection and assaying methods in which the presence of class M immunoglobulins (IgM) and class G immunoglobulins (IgG) that react with a bacterial antigen is detected, and preferably the amount of same is assayed. These determinations give more precise and complete information necessary for establishing the etiology and monitoring the progression of certain infectious diseases. Thus, in general, the IgMs appear earlier. As for the IgGs, they make it possible to establish the serological status of the patient with respect to a given bacterium, with a view to establishing the serological diagnosis of the infection or to establishing the degree of protection of the organism against this bacterium.

The specificity of the infectious antigen/serum antibody reaction conditions the specificity and the positive predictive value of the serological test based on this reaction, and any binding of a nonspecific antibody to the bacterial antigen limits the specificity and the predictive value of the serological test. The presence, in the test serum, of rheumatoid factors or antinuclear antibodies is a source of nonspecific binding of antibodies to the microbial antigen as explained hereinafter.

These rheumatoid factors are class M immunoglobulins which recognize the Fc fragment of the IgGs of various species, including human IgGs (anti-IgG IgM).

The presence of rheumatoid factors in the serum of a patient is responsible for false-positive results during the detection of IgMs specific for a microbial antigen in serological tests for the indirect diagnosis of infectious diseases. This is because these rheumatoid factors bind to the IgGs specific for the microbial antigen contained in the patient serum, and therefore appear to be falsely positive during the detection of specific IgMs in the serological reaction using the microbial antigen in agglutination detection tests.

Similarly, the presence, in the patient serum, of antinuclear antibodies limits the specificity of the microbial antigen-serum antibodies reaction. Antinuclear antibodies are in fact IgG-type antibodies directed nonspecifically against the assembly formed by DNA and the nuclear proteins of the chromosome of eukaryotic cells and of microorganisms, called histones. These antinuclear antibodies therefore bind nonspecifically to any eukaryotic cell, including fungi and parasites, and to any microorganism, bacterium, DNA virus, parasite or fungus. This phenomenon leads to a nonspecific positive reaction in serological tests using the detection of IgG specific for a bacterium, a DNA virus, a fungus or a whole parasite or comprising DNA/histone complexes as microbial antigen, by means of anti-IgG detection antibodies, in a serum containing antinuclear antibodies.

If the sample comprises rheumatoid factors (anti-IgG IgM), the latter may react with the immunoglobulins (IgG₁) attached to the solid support, so as to form the following complex with said first detection substance (anti-IgM*¹ Ab₁): (S-IgG₁-anti-IgG IgM-anti-IgM*¹ Ab₁) (S=solid support).

In one preferred embodiment,

1/ —the following pre-steps are carried out, in which:
said serum sample to be tested is brought into contact with said first and/or second detection substances and at least one said solid support to which has also been attached at least one control antigen among the following control antigens:
   a first control antigen corresponding to a nonspecific IgG-type immunoglobulin of the species of the patient, and
   a second control antigen comprising DNA/histone complexes, preferably all or part of nucleated cells comprising nuclei of nucleated cells, more preferably cells in a continuous line, and
   as appropriate, a third control antigen corresponding to a nonspecific IgM-type immunoglobulin of the species of the patient; the presence of said third control antigen being necessary in the case of the assaying of IgM, and
   a fourth control antigen containing protein A from a Staphylococcus aureus bacterium, said fourth antigen preferably being a whole Staphylococcus bacterium, and
at least one pre-control is carried out, preferably the series of following controls, comprising:
   a—the control of the reactivity of said first detection substance by verifying whether said first control antigen reacts with said first detection substance in the case of the assaying of IgG, and, as appropriate, the control of the presence of rheumatoid factors in said serum sample by verifying whether the first control antigen reacts with said serum sample and said second detection substance, in the case of the assaying of IgM,
   b—the control of the presence of antinuclear antibodies of said serum sample to be tested, by verifying whether said second control antigen reacts with said serum sample and the first detection substance, in the case of the assaying of IgG,
   c—the control of the reactivity of said second detection substance by verifying whether said third control antigen reacts with said first detection substance, in the case of the assaying of IgM, and
   d—the control of the presence of a human serum in the sample to be tested, and

2/ —the result of a reaction between said bacterial antigen, said serum sample and a said first and/or second detection substance is taken into account only if the control of the presence of a human serum is positive and if the following cumulative conditions are met, establishing the absence of antinuclear antibodies and the reactivity of said first and, as appropriate, second detection substances and, as appropriate, of rheumatoid factor:
a—said first control antigen reacts with said first detection substance,
b—said second control antigen does not react, and
c—as appropriate, if said third control antigen reacts with said second detection substance, in the case of the assaying of IgM, and
d—said fourth control antigen reacts with a said first and/or second detection substance, this first and/or second detection substance being one (or more) substance(s) which does (do) not react with said fourth control antigen.

When the absence of rheumatoid factor is established, the detection of a reaction between said first detection substance and said bacterial antigen (Agmic) is actually the evidence of the presence, in the serum tested, of class M immunoglobulins specific for the bacterial antigen (anti-Agmic IgM) through formation of the complex (S-Agmic-anti-Agmic IgM-anti-IgM*¹ Ab₁), and not of the presence of IgG specific for said bacterial antigen (anti-Agmic IgG), which could in fact form, in the presence of rheumatoid factor, a false-positive complex (S-Agmic-anti-Agmic IgG-anti-IgG IgM-anti-IgM*¹ Ab₁).

In addition, as explained hereinafter, when said first control antigen is constituted of an IgG of the species of the patient, in particular a human IgG, this makes it possible to verify the presence and the reactivity of a said first detection substance which specifically recognizes the IgGs in the serum of the species of the patient.

In the case of a human patient, nonconfluent human fibroblast cells in suspension, in particular HL60 cells, can advantageously be used as said second control antigen.

If the sample to be tested comprises antinuclear antibodies (which are IgGs, in particular human IgGs), they may react with said second control antigen (Ag₂) and be detected by said first detection substance (anti-IgG*² Ab₂) since the latter is a substance which reacts with IgGs of the species of the patient, in particular human IgGs, and form the complex (S-Ag₂-anti-Nucl IgG-anti-IgG*² Ab₂). The detection of a reaction between said first detection substance and said second control antigen (Ag₂) attached to a solid support necessarily signifies that a following complex with antinuclear antibodies (anti-nucl. Ab) has formed: (S-Ag₂-anti-nucl. IgG-anti-IgG IgM-anti-IgM*¹ Abᵢ), and therefore that the sample comprises both the rheumatoid factor and antinuclear antibodies.

Once the absence of antinuclear antibodies is established, the detection of a reaction of said second labeling substance with said microbial antigen is indeed the evidence of the presence of IgG specific for said bacterial antigen and of formation of a complex (S-Agmic-anti-Agmic IgG-anti-IgG*² Ab₂), and not of a false-positive complex resulting from the reaction of the antinuclear antibodies with the microbial antigen according to the complex (S-Agmic-anti-nucl. Ab-anti-IgG*² Ab₂).

The reactivity of said first detection substance introduced into said serum sample to be tested, in the absence of rheumatoid factor and of antinuclear antibodies in said sample, is also verified. This is because, if said first detection substance is indeed reactive in the absence of rheumatoid factor, the following complex: (S-IgG₁-anti-IgG*² Ab₂) should be detected on said first control antigen (IgG₁). In this case, the absence of reaction of said first detection substance with said second antigen is indeed the evidence of the absence of antinuclear antibodies.

If said second detection substance is present and is indeed reactive, a complex S-IgM₁-anti-IgM*¹ Ab₁ should be detected on said third control antigen (IgM₁). Consequently, the absence of detection of a complex comprising said second label at the level of said first control antigen (IgG₁) and, as appropriate, at the level of said second control antigen attached to a solid support, is indeed the evidence of the absence of rheumatoid factor.

More particularly, an automated deposit of a solution of IgG and of IgM of the species of the patient, in particular human IgG and IgM, which are γ-specific (specific for the gamma chain of the immunoglobulins of the species of the patient, in particular human immunoglobulins), as said first and third control antigens, is carried out.

The present invention therefore enables the systematic detection of rheumatoid factors and of antinuclear antibodies and the systematic control of the reactivity of the anti-immunoglobulin detection antibodies, in a serum used for a serological diagnosis by indirect immunofluorescence, after the automated deposit, on a solid support, of class G and M immunoglobulins of the species of the patient, in particular human immunoglobulins, and of nucleated cells of the species of the patient.

Another frequent error in carrying out the serological tests, in particular for serological tests carried out as a battery on a large number of sera to be tested, is due to faults in the introduction of the sera to be tested, in particular by pipetting. These errors occur in particular in the steps which involve moving the sample to be tested, in particular by pipetting; some containers, in particular containing the solid support on which the antigen to be detected is deposited, may inadvertently not be filled with the serum of the species of the patient, in particular human species, to be tested. It is known that the pipetting of serum is marred by a risk of errors of 0.1%, linked to a purely technical problem through a lack of pipetting by the pipette, or to a human error through a lack of pipetting by mistake.

These errors mean that it is necessary to introduce controls into the carrying out of the reaction. The systematic incorporation, during each new manipulation, of a negative control serum, i.e. a serum which does not contain antibodies specific for the antigen to be tested, makes it possible to interpret the positive reactions. Similarly, the incorporation of a positive control serum, i.e. a serum containing the antibody specific for the antigen tested, at a known titer, makes it possible to verify the quality of the antigen and of the immunoglobulin conjugated.

Insofar as protein A reacts with animal and human immunoglobulins nonspecifically, even in the case of a significant infectious pathological condition, it is possible to use this protein A as a positive control for the introduction of a serum of the species of the patient, in particular human species, in the sample to be tested.

More specifically, according to the present invention, the fact that said sample tested indeed contains a serum of the species of the patient is controlled by detecting whether immunoglobulins of the species of the patient react with a fourth control antigen containing protein A from a Staphylococcus aureus bacterium, said fourth antigen preferably being a whole Staphylococcus bacterium, by bringing said sample into contact with a solid support to which a said fourth control antigen is attached, in the presence of said second detection substance which is a substance that reacts with an immunoglobulin of the species of the patient and does not react with said fourth control antigen, preferably an antibody against the immunoglobulins of the species of the patient and which does not react with said fourth control antigen; the control of the presence of a serum is positive if said fourth antigen reacts with said serum sample and said first detection substance.

In one advantageous embodiment, said fourth control antigen is a whole Staphylococcus aureus bacterium comprising protein A. Use may more particularly be made of the Staphylococcus aureus bacteria deposited in public collections, such as the bacteria deposited with the A.T.C.C. under No. 29213 and with the C.N.C.M. [National Collection of Microorganism Cultures] of the Institut Pasteur (France) under number 65.8T, as described in the publication mentioned above [Rolain J M, Lecam C, Raoult D. Simplified serological diagnosis of endocarditis due to Coxiella burnetii and Bartonella. Clin. Diag. Lab. Immunol. 2003; 10:1147-8].

Moreover, in addition to the standard strains, any bacterial strain identified as Staphylococcus aureus may be used as said fourth control antigen.

Advantageously, use is made of a single solid support brought into contact with, as appropriate simultaneously or successively, said first and second detection substances comprising a first and, respectively, a second labeling element, the second labeling element emitting a signal different than the first labeling element, said first and second detection substances preferably comprising a first and, respectively, a second antibody which react only with a said class G and, respectively, class M immunoglobulin of the species of the patient.

In one embodiment of a method according to the invention, the following protocol of a succession of controls is therefore carried out:

1) It is verified that said fourth control antigen containing protein A reacts with said first detection substance. If it does not, the test is stopped, i.e. this sample is not taken into account.

2) If said first control antigen (IgG₁) reacts with said second detection substance (anti-IgM*¹ Ab1), the serum sample comprises rheumatoid factors, and therefore, here again, the tests of detection and of quantification of specific IgMs are not taken into account.

3) If said first control antigen does not react with the first detection substance (anti-IgG*² Ab₂), said first detection substance is not present or is not reactive. The result of the test concerning the detection of IgGs reacting with said bacterial antigens is not taken into account.

4) If said first control antigen reacts with the first detection substance, there is no rheumatoid factor and said first substance is reactive: the test can be continued, i.e. the results can be taken into account, with the proviso that the following verifications are carried out concerning the reactions with the second, third and fourth control antigens.

5) If said second control antigen containing a DNA/histone complex reacts with said first detection substance, antinuclear antibodies are present and the tests for detection and quantification of specific IgGs are not taken into account.

6) If said second control antigen reacts with said second detection substance, rheumatoid factors and antinuclear antibodies are present and the test is stopped.

7) If said second control antigen does not react, and if said first and second detection substances are present and reactive, there are no antinuclear antibodies and the test can be continued, with the proviso that the following verification is carried out.

8) It is verified that said third control antigen reacts with said second detection substance. If said third control antigen (IgM) does not react, said second detection substance is not present or does not react, and the test is stopped.

In summary, the result of the reaction with said microbial antigen is taken into account only if the following cumulative conditions are met:
said fourth control antigen reacts,
said first control antigen reacts with said first detection substance,
said second control antigen does not react, and
said third control antigen reacts with said second detection substance.

In one particular advantageous embodiment, an assay is carried out in which:

1—the five said bacteria or their bacterial antigens are deposited on a solid support,

2—said bacteria or their bacterial antigens are reacted with a serum from said patient, diluted to 1/16th, to which an anti-human IgG (anti-IgM) immunoglobulin, which is preferably labeled, has been added,

3—it is verified that said anti-human IgG (anti-IgM) immunoglobulins, which are preferably labeled, bind to said solid support.

In other words, if, after washing of said solid support, said labeled anti-IgG (anti-IgM) immunoglobulins are detected attached to said support, the binding thereof to said solid support could only have taken place by means of the IgG (IgM) antibodies (Ab) directed against said bacterium or corresponding bacterial antigen (Ag), by forming a complex: Ag-Ab-anti-IgG (anti-IgM) Ig.

In one particular embodiment, the following bacteria are deposited on said solid support:
a Borrelia burgdorferi, strain B31, bacterium;
a Leptospira interrogans serovar Icterohaemorrhagiae bacterium;
a Leptospira biflexa serovar Patoc bacterium;
a Borrelia recurrentis bacterium;
a Borrelia duttonii bacterium;
a p17 antigen of a Treponema pallidum bacterium and cardiolipid for syphilis serology.

As a solid support, use may be made of any device suitable for the handling of bacterial suspensions, and in particular tubes, glass slides, shell vials or rigid microtitration plates made of polyethylene, polystyrene, polyvinyl chloride or nitrocellulose, comprising microwells, glass slides being preferred.

As another labeling element for said detection substances, enzymatic labeling may also be used.

The expression “enzymatic labeling” signifies that the specific antibody is coupled to or complexed with an enzyme which, combined with the use of suitable reagents, enables a quantitative measurement of this specific antibody.

The substrate and the reagents are chosen such that the final product of the reaction or of the sequence of reactions brought about by the enzyme and using these substances is:
either a colored or fluorescent substance which diffuses in the liquid medium surrounding the sample tested and which is the subject either of the final spectrophotometric or fluorimetric measurement, respectively, or of an evaluation by eye, optionally relative to a range of calibrated shades,
or an insoluble colored substance which is deposited on the sample tested and which may be the subject either of a measurement by reflectance photometry or of an evaluation by eye, optionally relative to a range of calibrated shades.

When a detection substance that has been made fluorescent is used, the fluorescence associated with the sample tested is read directly on a suitable instrument capable of detecting the radiation at the emission wavelength and of quantifying it.

The expression “fluorescent labeling” signifies that the antibody has been made fluorescent by coupling or complexation with a suitable fluorescent agent such as fluorescein iso(thio)cyanate.

When an enzyme is used on the specific antibody, the appearance of a colored or fluorescent product is obtained by adding a solution containing the substrate for the enzyme and one or more secondary reagents for finally obtaining, as reaction product, either a colored product that is soluble in the medium, or an insoluble colored product, or a soluble fluorescent product, as was explained above. The light signal originating from the samples thus treated is then measured using the instrument suitable for each case: transmission photometer, reflectance photometer or fluorimeter, respectively. Alternatively, the coloration obtained may also be evaluated by eye, optionally with the aid of a range of calibrated colored solutions.

If alkaline phosphatase is used as the enzyme, the coupling of this enzyme with the specific antibody is carried out according to the method proposed by Boehringer Mannheim-Biochemica. The preferred substrates for this enzyme are para-nitrophenylphosphate for fluorimetric reading or bromo-5-chloro-4-indolyl-6-phosphate for obtaining an insoluble colored reaction product. β-D-galactopyranoside or methyl-4-umbelliferyl-β-D-galactopyranoside may likewise be used as enzyme.

Preferably, the specific antibodies can be coupled to peroxidase. In this case, the coupling method is derived from that described by M. B. Wilson and P. K. Nakane in Immunofluorescence and related staining techniques, W. Knapp, K. Kolubar, G. Wicks ed. Elsevier/North Holland. Amsterdam 1978, p. 215-224.

The reagents used to reveal the peroxidase conjugated to the specific antibodies contain aqueous hydrogen peroxide, a substrate for the enzyme and a suitable chromogen, for example ortho-phenylenediamine or azino-2-2′-bis(3-ethylhiazoline-6-sulfonic acid) or ABTS, in order to obtain a colored final reaction product which is soluble in the medium, or alternatively 3,3′-diaminobenzidine or 3-amino-9-ethylcarbazole or 4α-chloronaphthol, in order to obtain an insoluble final reaction product, or alternatively para-hydroxyphenylpropionic acid in order to obtain a fluorescent reaction product which is soluble in the medium.

Another embodiment of the invention is the use of immunoglobulin coupled to acetylcholinesterase.

The acetylcholinesterase is coupled to the antibody preferably using a method derived from that described in French patent No. 2 550 799, or a method which comprises, schematically, preparing fragments of the antibody by means of a known technique, modifying the enzyme by reaction with a suitable heterobifunctional agent and, finally, coupling the products thus obtained. Other known methods for constructing immunoenzymatic conjugates may also be used in this case.

The revealing of the enzymatic activity specifically linked to the antigen recognized by the acetylcholinesterase conjugate is preferably carried out according to the well-known technique which uses acetylthiocholine as enzyme substrate and the Ellman reagent, or 5,5′-dithio-2-nitrobenzoic acid as chromogen, according to any variant suitable for the case examined, for example that described by Pradelles et al., in Anal. Chem. 1985; 57:1170-1173.

The chromogens mentioned are used as they are or in the form of water-soluble salts.

As explained in WO2005/064340, conditions for depositing said control antigens and bacterial antigens on a solid support, which make it possible to just use deposition by simple physical adsorption, more particularly when said antigens are corpuscular antigens, are known.

Advantageously, said control antigens and corpuscular bacterial antigens are deposited as a mixture with a protein binder, which stabilizes the attachment to said solid support.

More particularly, said protein binder is chosen from the complex organic mixture formed by egg yolk, gelatin, bovine serum albumin or a nonhuman, preferably goat, polyclonal IgG.

These protein binders operate as a biological adhesive for said antigen on the solid support.

The present invention also provides a diagnostic kit that is of use for implementing a method according to the invention, comprising:
a said same solid support to which is attached at least one said plurality of bacterial antigens, and preferably at least one said control antigen, and
reagents such as a first and/or second said detection substance and reagents that are of use for detecting said first and/or second labeling elements.

Advantageously, in the methods and diagnostic kits according to the invention, a glass or plastic slide, a titration tube or a well of a microtitration plate made of plastic is used as a solid support, and more particularly, any device suitable for handling cellular and bacterial suspensions, and in particular tubes, glass or polymer slides, “shell” vials or rigid microtitration plates made of polyethylene, polystyrene or chloride, may be used as a solid support.

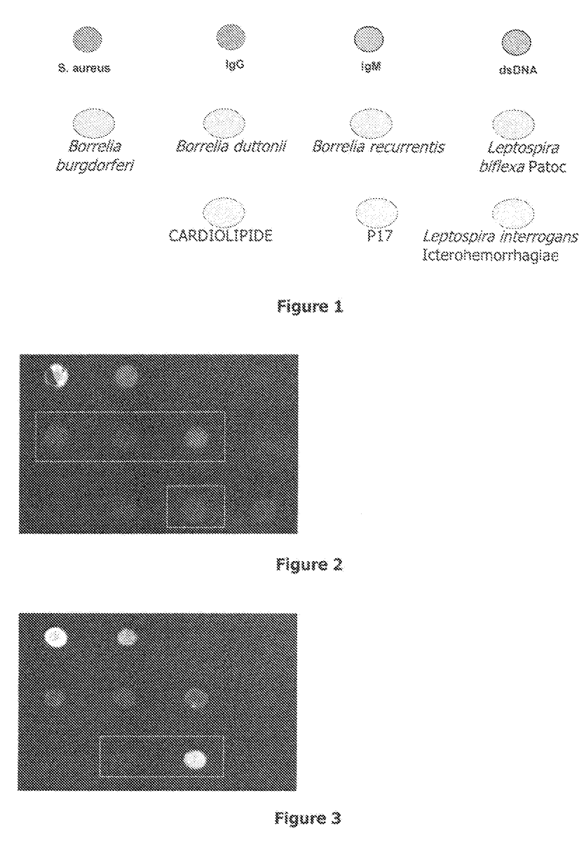

Other characteristics and advantages of the present invention will emerge on reading the detailed disclosure of the example which follows, which describes the detailed experiment with the aim of carrying out the invention, and which is given purely by way of illustration, with reference to FIGS. 1 to 3 in which:

FIG. 1 represents the scheme of a spirochete multiplexed slide comprising seven spirochete antigens and four control antigens;

FIG. 2 represents the reactions of a borreliosis serum between the three Borrelia antigens and the P17 serology of a slide of FIG. 1 for IgGs, and

FIG. 3 represents the reaction of a serum of a progressive syphilis for IgGs with a slide of FIG. 1.

### EXAMPLE

### 1—Antigens

The bacteria Borrelia burgdorferi, Leptospira interrogans, Leptospira biflexa serovar Patoc, Borrelia recurrentis, Borrelia duttonii and Borrelia crocidurae and the P17 antigens of Treponema pallidum and cardiolipid are accessible to the public through various sources. They have been described in the literature, and have been deposited in various deposit collections, in particular the bacterium Borrelia burgdorferi strain B31 deposited with ATCC under number ATCC 35210, Leptospira biflexa serovar Patoc under number ATCC 23582, and Leptospira interrogans serovar Icterohaemorrhagiae in the collection of the National Reference Center (Institut Pasteur, Paris). The bacteria Borrelia duttonii strain Ly [Cutler S J et al. Int. J. Syst. Bacteriol. 1999; 49:1793-1799], Borrelia recurrentis strain A1 [Cutler S J et al. Lancet. 1994; 343:242], and Borrelia crocidurae are available in the collection of the National Reference Center (Institut Pasteur, Paris) and have been deposited in the Collection de souches de l'Unité des Rickettsies [Reckettsia Unit Strain Collection], recognized by the World Data Center for Microorganisms (http://wdcm.nig.ac.jp) under the number WDCM 875, respectively under the numbers CSURP-1, CSURP-2 and CSUR-3. Borrelia duttonii is used as a representative of tick-borne forms of borreliosis, other than Lyme disease. The recombinant P17 antigen of Treponema pallidum is obtained from Fitzgerald (Fitzgerald, Conrad, Mass., USA) under the reference number 30-AT68 and the cardiolipid is obtained from Sigma-Aldrich (Saint-Quentin Falavier, France) under the reference number C-1649.

Borrelia burgdorferi strain B31 (ATCC 35210), Borrelia recurrentis strain A1 and Borrelia duttonii strain Ly were used as antigens for Borrelia. These antigens are obtained as follows: the bacteria are cultured in a BSK-H broth (reference BB291, Sigma-Aldrich, Saint-Quentin Falavier, France) at 33° C. and their density is assessed by microscopic observation in the fresh state. Leptospira biflexa serovar Patoc (ATCC 2374) and Leptospira interrogans serovar Icterohaemorrhagiae were used as antigens for leptospira. These bacteria are cultured in a leptospira medium (reference 55954 F, Biorad, Marnes-la-Coquette, France) at 30° C. in the dark, and their density is assessed by microscopic observation in the fresh state.

After purification, the bacteria are concentrated appropriately (the concentration is measured through the protein concentration), made fluorescent by adding AMCA and added to a suitable biological adhesive and then deposited, using a spotter, on the solid support. The concentration of cardiolipid and of P17 protein is adjusted, and then these antigens are mixed, at an appropriate concentration, with a biological adhesive previously made fluorescent by adding AMCA. Finally, the slide thus constituted is fixed by means of a chemical process in order to stabilize the deposits on the slide, as described in WO 2005/064340.

The following control antigens were used:
as said fourth control antigen, Staphylococcus aureus deposited in the public collections such as the A.T.C.C. under No. 29213 and with the C.N.C.M. [National Microorganism Culture Collection] of the Institut Pasteur (France) under number 65.8T, as described in the publication mentioned above [Rolain J M, Lecam C, Raoult D. Simplified serological diagnosis of endocarditis due to Coxiella burnetii and Bartonella. Clin. Diag. Lab. Immunol. 2003; 10:1147-8],
as said first control antigen, a human IgG provided by the company Serotec Ltd (ref PHP 001),
as said third control antigen, human IgMs provided by the company Sigma Aldrich Chimie (ref I8260), and
as said second control antigen, dsDNA provided by the company Diarect (reference 12300).

FIG. 1 represents the slide with the four control antigens and six bacterial antigens and cardiolipid mentioned above.

### 2—Serological Technique

The technique used is the multiplexed indirect immunofluorescence technique. The inventors used materials and methods, and in particular detection immunoglobulins with fluorescent labels and an automated installation developed by the company INODIAG as described on the site www.inodiag.com and in WO2005/064340, comprising an incubator, a fluorescence reader and interpretation software for the incubation. The reading and the interpretation were based on spirochete multiplexed slides.

The scheme of a spirochete multiplexed slide is shown in FIG. 1. This slide in FIG. 1 comprises, in addition to the seven spirochete antigens, the four control antigens for verifying the bringing into contact of the slide with the patient serum to be tested, for verifying the quality of the anti-IgG and anti-IgM fluorescent conjugates used and for detecting the presence of antinuclear antibodies and of rheumatoid factors in the patient serum. The latter two types of antibodies are factors known to give a false-positive serological reaction.

In this example, each of the sera tested was diluted to 1:16 and was then incubated in the presence of a spirochete multiplexed slide. The measurement comprised the measurement of fluorescence associated with a conjugated anti-human IgG antibody and the measurement of fluorescence associated with an anti-human IgM antibody according to the methods previously described in WO 2005/064340.

For each specific antigen, a fluorescence cut-off giving a specificity of at least 95% and a fluorescence cut-off giving a sensitivity of at least 95% were determined using 10 negative control sera and 10 positive sera in a reference method. For some measurements, these two cut-offs are the same.

With the detection substances and the instrument and the calculation software used, the following cut-off values were determined.

For the Borrelia duttonii and Borrelia recurrentis specific antigens, the two cut-offs used are those determined for Borrelia burgdorferi. The fluorescence cut-off values used in this study are 2500-3500 units of fluorescence for the borreliae with respect to IgG and 2000 units of fluorescence for the borreliae with respect to IgM, 2000-2500 units of fluorescence for Leptospira biflexa Patoc IgG and IgM, 3000 units of fluorescence for Leptospira interrogans Icterohemorraghiae IgG, 2000 units of fluorescence for Leptospira interrogans Icterohaemorrhagiae IgM, 500-800 units of fluorescence for cardiolipid IgG, 1000-1400 units of fluorescence for cardiolipid IgM, and 1000-1500 units of fluorescence for p17 IgG. CL 3—Sera Tested

In order to be able to set up the technique, the inventors selected a certain number of sera from patients having presented an identified pathological condition. The inventors tested a total of 211 sera, comprising 19 patients having Lyme disease (Borrelia burgdorferi) with the presence of specific antibodies detected by ELISA and confirmed by Western blotting; 17 sera from patients having a positive serology for Leptospira patoc by immunofluorescence; 34 sera from patients having syphilis with the presence of anti-Treponema pallidum antibodies and a positive Veneral Disease Research Laboratory (VDRL) test; 51 sera from patients who were febrile upon returning from an overseas country; 20 sera from patients having uveitis; 51 sera taken from patients who were febrile after being bitten by a tick in Senegal and were therefore suspected of a Borrelia crocidurae infection; and 17 negative control sera having no antibodies against syphilis (Treponema pallidum).

Results

Out of 211 samples:
8 samples were discarded for a serum fault or a detection substance fault or poor incubations,
19 samples contained sera with the presence of rheumatoid factors (definition of the alert “presence of rheumatoid factors”: Ig G at 594 nm>5000 units of fluorescence),
3 samples contained sera with antinuclear antibodies,
4 samples contained sera with rheumatoid factors and antinuclear antibodies.

In total, 34 sera were therefore excluded from the analysis because they were detected by a software alarm signal.

The analysis therefore related to 177 samples.

Among these 177 sera, 53 are negative for all the parameters studied: Borrelia burgdorferi IgG and IgM, Borrelia duttonii IgG and IgM, Borrelia recurrentis IgG and IgM, Leptospira biflexa Patoc, Leptospira interrogans Icterohemorraghiae, cardiolipid and p17, and 124 sera are positive with respect to one or more parameters.

Among the 124 positive sera, 56 are monospecific, i.e. exhibit a response with a fluorescence signal greater than the specificity cut-off for the reaction with a bacterial antigen of the support, and 68 sera exhibit only cross-reactions, i.e. reaction signals with strengths between the two sensitivity and specificity cut-offs.

All the possible cross-reactions were observed.

When the analysis is carried out for each category of sera tested, the results are the following:

1) Among the 18 interpretable sera from patients having a Borrelia burgdorferi serology:
9 sera are negative (no signal above the sensitivity cut-offs), making it possible to conclude that spirochetes are absent;
7 sera exhibit only cross-reactivity, in serological tests, with the other Borrelia or p17, making it possible to conclude that bacteria of the Borrelia genus other than Borrelia burgdorferi, Borrelia recurrentis or Borrelia duttonii are present, and
2 sera are monospecific with respect to Borrelia burgdorferi and, respectively, Treponema pallidum (a single signal from a reaction with a bacterial antigen above the specificity cut-off, and signals from cross-reactions);

2) among the 34 interpretable sera from patients having a serology positive for syphilis by means of the reference methods,
17 are confirmed by the multiplexed serology with signals from a reaction with p17 and, in certain cases, cardiolipid, which are greater than the specificity cut-offs in question, and
17 have cross-reactivities, in serological tests, with Borrelia burgdorferi, the other Borrelia and Leptospira biflexa Patoc, making it possible to conclude that a nonpathogenic Leptospira or Borrelia spirochete bacterium other than those tested is present, but without determination of the genus or the species;

3) among the 8 interpretable sera having a serology positive for Leptospira spp. by means of the reference method,
3 are confirmed by the multiplexed serology,
1 is identified as Borrelia burgdorferi (single reaction signal greater than the specificity cut-off), and
4 exhibit solely cross-reactivity, in serological tests, only with the Borrelia, making it possible to conclude that there is a borreliosis other than those tested, without determination of the species;

4) among the 44 interpretable sera taken from patients having been bitten by a tick in Senegal, suspected of having been exposed to Borrelia crocidurae,
12 are negative with respect to all spirochetes,
6 are diagnosed as having syphilis, i.e. are monospecific-positive for syphilis, and
1 is monospecific-positive for leptospirosis (only L. interrogans reacting with a signal greater than the specificity cut-off), and
25 cross with all the Borrelia and Leptospira, making it possible to conclude that a Borrelia or Leptospira is present, without determination of the genus or the species;

5) among the 43 interpretable sera taken from patients who were febrile upon returning from the tropics,
12 are negative for all spirochetes,
13 are monospecific-positive for syphilis,
1 is monospecific-positive for Borrelia burgdorferi, and
17 have cross-reactivities, in serological tests, which cross with all the spirochetes, making it possible to conclude that a Borrelia or Leptospira is present, without determination of the genus or the species;

6) among the 20 interpretable sera taken from patients having uveitis, 12 are negative, 3 have a p17 serology, 1 has Borrelia duttonii IgMs, 1 has Borrelia burgdorferi IgGs and 3 have cross-reactivities, in serological tests, between Borrelia burgdorferi, Borrelia recurrentis and p17;

7) among the 16 interpretable negative-control sera,
10 are actually negative, and
4, however, have a p17-positive serology, including 1 active syphilis (reaction with cardiolipid), and
1 is monospecific-positive for Borrelia burgdorferi, and
7 have only cross-reactivities, in serological tests, between Leptospira biflexa patoc and p17, making it possible to conclude that a Borrelia or a nonpathogenic leptospira of the Leptopsira biflexa species, other than those tested, is present, without determination of the species.

Overall, the use of the multiplexed spirochete serology system made it possible to demonstrate the serological reactions that were definitely monospecific and the serological cross-reactions, and therefore to increase the positive predictive value of the spirochete serologies. In addition, new serological cross-reactions were demonstrated, as is illustrated in table 2.

These results are illustrated in FIGS. 2 and 3 which follow, showing the image of the slide after incubation with the serum and the conjugated antibodies.

FIG. 2 represents the reactions of a borreliosis serum between the three Borrelia and the P17 serology for IgGs.

It is a “return from the topics” serum given a positive titer for Borrelia burgdorferi by the reference method (ELISA 0.61/cut-off 0.521). With the above technique, a response which is strongly positive for Borrelia recurrentis (fluorescence value of 8330 for cut-offs of 2500-3500) and Borrelia burgdorferi (fluorescence value of 2619 for cut-offs of 2500-3500), intermediate positive (cross-reactions) for Borrelia burgdorferi (fluorescence value of 2619 for cut-offs of 2500-3500) and Borrelia duttonii (2688 for cut-offs of 2500-3500) and positive P17 (fluorescence value of 1261, cut-off 1000-1500) is found. The interpretation of this serology is therefore a borreliosis caused by Borrelia recurrentis.

FIG. 3 represents the reaction of a progressive syphilis serum for IgGs.

It is a serum given as VDRL-TPPA positive. With the above technique, it is found to be strongly positive for p17 (12 210 for an upper cut-off at 1500), and positive for cardiolipid (value of 1076 for an upper cut-off at 700). This serum exhibits cross-reactions with Borrelia burgdorferi (3022 for an upper cut-off at 3500) and Borrelia recurrentis (3253 for an upper cut-off at 3500). The interpretation of this serology is therefore the presence of a progressive syphilis.

Interpretation

These results illustrate the ability of the invention to detect, by virtue of the multiplexing, on the one hand, antibodies specific for each of the spirochete species tested and, on the other hand, the presence of cross-reactivities, in serological tests, in particular cross-reactivities, in serological tests, which have not been reported in the literature. In particular, the inventors demonstrate serological cross-reactions among the borreliae, between, on the one hand, Borrelia burgdorferi (Lyme disease) and, on the other hand, Borrelia duttonii and Borrelia recurrentis. This is a new piece of data, since a single published study had detected, in 40 sera from patients having Lyme disease (Borrelia burgdorferi), 80% false positives against a tick-borne relapsing fever agent, Borrelia hermsii [Magnarelli L A, Anderson J F, Johson R C. Cross-reactivity in serological tests for Lyme disease and other spirochetal infections. J. Infect. Dis. 1987; 156:183-188]. The data found by the inventors confirm the prior data, concerning the false-positive reactions against B. burgdorferi of sera from patients with tick-borne or louse-borne relapsing fever [Magnarelli L A, Anderson J F, Johson R C. Cross-reactivity in serological tests for Lyme disease and other spirochetal infections. J. Infect. Dis. 1987; 156:183-188]. These results make it possible to understand the results previously published by other teams on the presence of Lyme disease in Africa where the spirochete responsible has, however, never been detected [Jodi J O, Gathua S N. Lyme disease: report of two cases. East. Afr. Med. J. 2005; 82:267-9]. These serological diagnosis most probably correspond to cross-reactions between borreliae. In addition, the inventors demonstrate, for the first time, cross-reactions between on the one hand, Treponema pallidum (syphilis) and, on the other hand, Borrelia duttonii and Borrelia recurrentis [Norris S J. et al. Treponema and other human host-associated spirochetes, In: Manual of Clinical Microbiology 8th Edition. Murray P R, Baron E J, Jorgensen J H, Pfaller M A, Yolken R H (Eds). ASM Press, Washington D.C., 2003 pp. 955-971]. In fact, in the study by LA Magnarelli and collaborators, 1 serum out of 15 sera from patients with syphilis reacted against Borrelia hermsii [Magnarelli L A, Anderson J F, Johson R C. Cross-reactivity in serological tests for Lyme disease and other spirochetal infections. J. Infect. Dis. 1987; 156:183-188]. Finally, the inventors demonstrate, for the first time, cross-reactions, in serological tests, between, on the one hand, Leptospira sp. and, on the other hand, Borrelia duttonii and Borrelia recurrentis.

TABLE 1
Serological cross-reactions by indirect immunofluorescence, reported in the literature

| | Cross-reactions | | | | |
|---|---|---|---|---|---|
| | | | | Tick-borne | |
| Etiology | T. pallidum | L. interrogans | B. burgdorferi | borreliosis | B. recurrentis |
| T. pallidum | | | + ^{1, 2} | | |
| L. interrogans | | | + ^{1, 3} | | |
| B. burgdorferi | + ⁴ | | | | |
| Tick-borne | | | + | | |
| borreliosis | | | | | |
| B. recurrentis | | | + | | |

References:
¹ Magnarelli L A. et al. J. Infect. Dis 1987; 156: 183-8
² Raoult D. et al J. Clin. Microbiol 1989; 27: 2152-5
³ Raoult D. et al Presse Med. 1988; 17: 485
⁴ Norris S J. et al Manual of Clinical Microbiology 2003; 955-71

TABLE 2
Serological cross-reactions by indirect immunofluorescence,
observed by multiplexed spirochete serology

| | Cross-reactions | | | | | |
|---|---|---|---|---|---|---|
| Etiology | T. pallidum | L. interrogans | L. biflexa | B. burgdorferi | B. duttonii | B. recurrentis |
| T. pallidum | + | + | + | + | + | + |
| L. interrogans | + | + | + | + | + | + |
| L. biflexa | + | + | + | + | + | + |
| B. burgdorferi | + | + | + | + | + | + |
| B. duttonii | + | + | + | + | + | + |
| B. recurrentis | + | + | + | + | + | + |
| B. crocidurae | + | + | + | + | + | + |

1. A method for the serological diagnosis, in vitro, of an infection with a spirochete bacterium which is pathogenic in humans, chosen from bacteria of the Borrelia, Leptospira and Treponema genera, by indirect immunodetection, characterized in that the following steps are carried out, comprising:
1/bringing a same patient serum sample into contact with
a/ —a first detection substance and/or respectively second detection substance constituted of an antibody which reacts only with an immunoglobulin of the species of the patient of IgG type and/or respectively IgM type and
b/ —a plurality of bacterial antigens deposited on several zones of a same solid support, comprising:
a plurality of bacterial antigens of bacteria, of the Borrelia genus, of different species comprising at least the bacteria Borrelia burgdorferi, Borrelia recurrentis and a tick-borne Borrelia species other than Borrelia burgdorferi, chosen from Borrelia duttonii and Borrelia crocidurae, said bacterial antigens being constituted respectively of at least fractions of bacteria of said species, each said fraction of said bacterium comprising at least one specific antigen and one nonspecific antigen of said species of bacterium, preferably whole bacteria, and
a plurality of bacterial antigens of bacteria, of the Leptospira genus, of different species comprising at least one commensal species which is not pathogenic in humans and one pathogenic bacterium of the Leptospira interrogans species, said bacterial antigens being constituted respectively of at least fractions of bacteria of said species, each said fraction of said bacterium comprising at least one specific antigen and one nonspecific antigen of said bacterium, preferably whole bacteria, and
at least one specific bacterial antigen of the bacterium Treponema pallidum, and
2/detecting and preferably quantifying the serological reactions of IgG-type and/or IgM-type immunoglobulins present, as appropriate, in said patient serum, which react with at least one of said bacterial antigens, by detecting and preferably quantifying a signal emitted by a ternary complex of an immunological reaction between (a) said bacterial antigen(s), (b) a said immunoglobulin which reacts with at least one of said bacterial antigens, said immunoglobulin being an IgG-type immunoglobulin for detecting and assaying IgG, and/or respectively a said IgM-type immunoglobulin for detecting and assaying IgM, and (c) a said first detection substance for detecting and assaying IgG and/or respectively second detection substance for detecting and assaying IgM.

2. The method as claimed in claim 1, characterized in that the plurality of bacteria of the Borrelia genus of different species is constituted of the three bacteria Borrelia burgdorferi, Borrelia recurrentis and Borrelia duttonii.

3. The method as claimed in claim 1, characterized in that at least one assay of the IgM-type antibodies against the louse-borne Borrelia bacterium Borrelia recurrentis is carried out.

4. The method as claimed in either of claim 1, characterized in that the plurality of bacteria of the Leptospira genus is constituted of the two bacteria Leptospira biflexa serovar Patoc and Leptospira interrogans serovar Icterohaemorraghiae.

5. The method as claimed in one of claim 1, characterized in that the specific bacterial antigen of the Treponema pallidum bacterium is the p17 antigen.

6. The method as claimed in one of claim 1, characterized in that said solid support comprises an additional zone on which cardiolipid is deposited.

7. The method as claimed in claim 1, characterized in that the test is carried out for a single dilution of the serum sample, diluted to 1/16th.

8. The method as claimed in claim 1, characterized in that the following are determined:
the presence of a particular bacterium of a said species Borrelia burgdorferi or Borrelia recurrentis or a tick-borne Borrelia other than Borrelia burgdorferi, such as Borrelia duttonii, or a pathogenic bacterial species Leptospira interrogans or a nonpathogenic leptospiral bacterial species such as Leptospira biflexa, or the presence of Treponema pallidum, if there is observed a plurality of signals from the reactions with said bacterial antigens of bacteria of a same species, deposited on the solid support, preferably, as appropriate, of bacteria of the same genus, with a signal that is of significantly greater strength for said particular bacterium than for the others, and
the presence of a spirochete bacterium of the Borrelia genus or of the Leptospira genus, of a species other than those of the bacteria of said corresponding bacterial antigens deposited on the solid support, without determination of the species, if there is observed a plurality of signals from reactions with said bacterial antigens for bacteria of the same genus Borrelia or Leptospira, without a signal that is of significantly greater strength than the others for a said particular bacterium, and without a signal from reaction with a bacterial agent of a bacterium of the other genus Leptospira or, respectively, Borrelia, and
the presence of a spirochete bacterium of the Borrelia genus or of the Leptospira genus, other than those of the bacteria of said corresponding bacterial antigens deposited on the solid support, without determination of the genus Borrelia or Leptospira, if there is observed a plurality of signals from reactions with said bacterial antigens for bacteria of the two genera Borrelia and Leptospira and, optionally, in addition, with Treponema pallidum, without a signal that is of significantly greater strength than the others for a said particular bacterium, and
the absence of any spirochete bacterium of the Borrelia or Leptospira genus, if no signal is observed with any of said bacterial antigens of bacteria of the Borrelia and Leptospira genera, and
the absence of Treponema pallidum, if no signal from reactions with said corresponding bacterial antigen is observed.

9. The method as claimed in claim 8, characterized in that the presence of an active Treponema pallidum bacterium is determined if signals from a reaction with said Treponema bacterial antigen and with the cardiolipid are observed, said signals preferably being stronger than signals from cross-reactions with other said bacterial antigens.

10. The method as claimed in claim 1, characterized in that the absence of an active Treponema pallidum bacterium is determined if no signal from a reaction with the cardiolipid is obtained.

11. The method as claimed in claim 1, characterized in that:
1—detection signal strength cut-offs are determined for each of said bacterial antigens as deposited on said solid support, said cut-offs comprising:
a) a so-called “specificity” cut-off, starting from which it is considered that the signal with a said bacterial antigen may include the signal from a reaction with a said specific antigen, and
b) as appropriate, a so-called nonspecific “sensitivity” cut-off, such that
if the signal has a strength between those of said specificity cut-off and said sensitivity cut-off, it is considered that it may include a reaction with a said nonspecific antigen, and does not include a reaction with a said specific antigen, and
if the signal has a strength below that of said sensitivity cut-off, it is considered to be not significant, and
2—the following are determined:
a) the presence of a bacterium of the Borrelia genus or of the Leptospira genus, of a species other than those of the bacteria of said bacterial antigens deposited on the solid support, with determination of the genus but without determination of the species and, as appropriate, of the serovar, if there is observed a plurality of signals from reactions with said bacterial antigens of bacteria of the same genus Borrelia or Leptospira, deposited on the solid support, said signals having strengths greater than or equal to that of said sensitivity cut-off and below that of said specificity cut-off, no signal from a reaction with a said bacterial antigen deposited on the solid support having an intensity greater than or equal to that of said specificity cut-off, and no signal from a reaction with a said bacterial antigen of the other genus Leptospira or, respectively, Borrelia, deposited on the solid support, having a strength greater than or equal to that of said sensitivity cut-off, and
b) the presence of a bacterium of the Borrelia genus or of the Leptospira genus, and of a species other than those of the bacteria of said bacterial antigens deposited on the solid support, without determination of the genus or of the species, or, as appropriate, of the serovar, if there is observed a plurality of signals from reactions with said bacterial antigens of bacteria of the two genera Borrelia and Leptospira, deposited on the solid support, said signals having strengths greater than or equal to that of said sensitivity cut-off and below that of said specificity cut-off, and no signal from a reaction with a said bacterial antigen deposited on the solid support having a strength greater than or equal to that of said specificity cut-off, and
c) the presence of a particular bacterium of a said species Borrelia burgdorferi or Borrelia recurrentis or a tick-borne Borrelia other than Borrelia burgdorferi, such as Borrelia duttonii or Borrelia crocidurae, or a pathogenic bacterial species Leptospira interrogans or a nonpathogenic leptospiral bacterial species such as Leptospira biflexa, or the presence of Treponema pallidum, if there is observed a single signal from a reaction with a said bacterial agent of a particular bacterium of the same species, deposited on the solid support, which has a strength greater than or equal to that of the specificity cut-off, and, optionally, reaction signals having strengths below the specificity cut-offs for the reactions with the other bacterial agents deposited on the solid support, and
d) the presence of a Treponema pallidum bacterium if there is observed a signal from a reaction with the specific antigen of Treponema pallidum, deposited on the solid support, which has a strength greater than or equal to that of said specificity cut-off for reaction with a said bacterial agent of a said particular bacterium, and
e) the presence of an active Treponema pallidum bacterium if, in addition, there is observed a signal from a reaction with the cardiolipid, which has a strength greater than that of the sensitivity cut-off for said cardiolipid.

12. The method as claimed in claim 1, characterized in that said first and/or second detection substances comprise said first and/or, respectively, second labeling elements emitting signals that can be distinguished.

13. The method as claimed in claim 12, characterized in that said first and/or second detection substances are goat or chicken immunoglobulins, which are respectively anti-IgG and/or anti-IgM.

14. The method as claimed in claim 1, characterized in that said first and/or second detection substances comprise said first and/or, respectively, second labeling elements emitting fluorescent signals at different excitation wavelengths that can be quantified by automated reading of the strength of the fluorescent signal emitted by said fluorescent labeling element by means of a suitable reading instrument capable of quantifying it.

15. The method as claimed in claim 1, characterized in that:
1/ —the following pre-steps are carried out, in which:
said serum sample to be tested is brought into contact with said first and/or second detection substances and at least one said solid support to which has also been attached at least one control antigen among the following control antigens:
a first control antigen corresponding to a nonspecific IgG-type immunoglobulin of the species of the patient, and
a second control antigen comprising DNA/histone complexes, preferably all or part of nucleated cells comprising nuclei of nucleated cells, more preferably cells in a continuous line, and
as appropriate, a third control antigen corresponding to a nonspecific IgM-type immunoglobulin of the species of the patient; the presence of said third control antigen being necessary in the case of the assaying of IgM, and
a fourth control antigen containing protein A from a Staphylococcus aureus bacterium, said fourth antigen preferably being a whole Staphylococcus bacterium, and
at least one pre-control is carried out, preferably the series of following controls, comprising:
a—the control of the reactivity of said first detection substance by verifying whether said first control antigen reacts with said first detection substance in the case of the assaying of IgG, and, as appropriate, the control of the presence of rheumatoid factors in said serum sample by verifying whether the first control antigen reacts with said serum sample and said second detection substance, in the case of the assaying of IgM,
b—the control of the presence of antinuclear antibodies in said serum sample to be tested, by verifying whether said second control antigen reacts with said serum sample and the first detection substance, in the case of the assaying of IgG,
c—the control of the reactivity of said second detection substance by verifying whether said third control antigen reacts with said first detection substance, in the case of the assaying of IgM, and
d—the control of the presence of a human serum in the sample to be tested, and
2/ —the result of a reaction between said bacterial antigen, said serum sample and a said first and/or second detection substance is taken into account only if the control of the presence of a human serum is positive and if the following cumulative conditions are met, establishing the absence of antinuclear antibodies and the reactivity of said first and, as appropriate, second detection substances and, as appropriate, of rheumatoid factor:
a—said first control antigen reacts with said first detection substance,
b—said second control antigen does not react, and
c—as appropriate, if said third control antigen reacts with said second detection substance, in the case of the assaying of IgM, and
d—said fourth control antigen reacts with a said first and/or second detection substance, this first and/or second detection substance being one (or more) substance(s) which does (do) not react with said fourth control antigen.

16. A kit that is of use for implementing a method as claimed in claim 1, characterized in that it comprises:
a said same solid support to which is attached at least one said plurality of bacterial antigens, and preferably at least one said control antigen, and
reagents such as a first and/or second said detection substance and reagents that are of use for detecting said first and/or second labeling elements.
